(19) United States
(12) Patent Application Publication
(10) Pub. No.: US 2011/0243852 A1
(43) Pub. Date: Oct. 6, 2011

(54) METHOD FOR THE PRODUCTION OF LABELLED SCAFFOLDS, COMPRISING AT LEAST ONE REACTIVE FLUORINATED SURFACTANT, AND SCAFFOLD PRODUCED THEREWITH

(75) Inventors: Emiel Peeters, Eindhoven (NL); David Halter, Eindhoven (NL); Dirk Jan Broer, Geldrop (NL); Roel Penterman, Eindhoven (NL)

(73) Assignee: KONINKLIJKE PHILIPS ELECTRONICS N.V., EINDHOVEN (NL)

(21) Appl. No.: 13/139,361

(22) PCT Filed: Dec. 7, 2009
(86) PCT No.: PCT/IB2009/055543

§ 371 Date: Jun. 13, 2011

(30) Foreign Application Priority Data
Dec. 17, 2008 (EP) 08171969.2

(51) Int. Cl. A61K 49/00

(52) U.S. Cl. 424/9.1

(57) ABSTRACT

The present invention is related to scaffolds for tissue engineering and organ engineering. More particularly, the present invention is related to a method for the production of a labelled scaffold for tissue and/or organ engineering comprising a reactive fluorinated surfactant, which serve as imaging label for medical imaging means, like CT, MRI, PET, scintigraphy and/or ultrasound imaging and the like.

### FIELD OF THE INVENTION

The present invention is related to scaffolds for tissue engineering and organ engineering. More particularly, the present invention is related to a method for the production of a labelled scaffold for tissue and/or organ engineering comprising a reactive fluorinated surfactant, which serves as imaging label for medical imaging means, like CT, MRI, PET, scintigraphy and/or Ultrasound imaging and the like.

### BACKGROUND OF THE INVENTION

Regenerative medicine is a new upcoming field that aims to develop innovative medical therapies that will trigger and enable the body to repair, restore and regenerate damaged or diseased cells, tissues and organs. Therapies based on the principle of tissue regeneration will have a strong clinical and economical impact as they offer a significant improvement in life expectancy and quality of life for large groups of chronically ill patients.

Tissue engineering is a relatively young discipline which aims at producing, in the laboratory or in vivo, tissues, or organs, which may then be used to repair, or replace, defective tissues, or organs, of a patient.

In many cases, the said tissues, or organs, are being produced with help of a scaffold, this being a three-dimensional matrix which the cells use as basis for their growth, and division, either in vitro or in vivo. Such scaffold needs to mimic the in vivo milieu, and enable cells to influence their own microenvironment. In order to do so, it needs to allow cell attachment and migration, deliver and retain cells and biochemical factors, enable diffusion of vital cell nutrients and expressed products, and exert certain mechanical and biological influences to modify the behaviour of the cells.

To provide for a proper functioning of the implanted tissue it is desirable to be able to visually control the actual state of the scaffold. This can be of particular importance for monitoring the continuous bio-degradation of the scaffold and to assess the structural and mechanical properties during this degradation process. However, the cells growing on the scaffold as well as the scaffolds itself provide little, or even no, contrast compared to the surrounding tissues in clinically relevant imaging modalities such as CT, MRI, PET, scintigraphy and/or Ultrasound imaging, and can therefore hardly be visualized. Therefore, there is a need for tissue and/or organ engineering scaffolds where a contrast agent is linked to or included in the polymer matrix.

US2006/0204445 addresses this need and discloses a matrix having a three-dimensional ultrastructure of interconnected fibers and pores to permit cell attachment, and further comprising an image enhancing agent. Said image enhancing agent is an MRI imaging label based on lanthanides and/or transition elements. The matrix comprises biomaterials, such as collagen, elastin, fibrous proteins and/or polysaccharide, and/or a synthetic polymer, and is for example produced by electrospinning. The image enhancing agents are incorporated within, or on, the scaffold matrix before seeding with cells. When the colonized scaffold forms a tissue layer of cells and is ready for use, the growth, development, and remodeling of the artificial tissue can be monitored using the incorporated agents.

Although lanthanide complexes are commonly used as image enhancing agents in MRI they have a few disadvantages. They need direct contact with water to provide imaging contrast and free uncomplexed lanthanides ions in general show high toxicity. The high toxicity of the free ions is especially worrisome when the agents are included in tissue engineering scaffold which is implanted in the human body and stays there for long time periods.

A known alternative imaging agent in tissue and/or organ engineering scaffolds are highly fluorinated compounds. They serve e.g. as imaging enhancing agent in fluor19 MRI. Fluor atoms are not natural occurring in the human body and fluor19 MRI is very sensitive. In general, fluorinated compounds are very inert and show no to little toxicity. In particular perfluorooctylbromide (PFOB) is known to be inert and is commonly used as contrast agent in fluor19 MRI. The most straightforward way to introduce the PFOB in the tissue engineering scaffold is by mixing labeling molecules into the polymer solution used for preparing the scaffold, e.g. by means of electrospinning. The labeling molecules are therefore partly enclosed within the meshwork made of the electrospun fibers.

A disadvantage of highly fluorinated compounds is the poor solubility of these compounds in polymer mixtures commonly used for tissue engineering scaffold preparation. This poor solubility leads to phase separation and inhomogeneous solutions for scaffold production.

Another disadvantage is the high evaporation rate of highly fluorinated compounds often resulting in complete evaporation of the fluorinated compound during processing of the polymer mixture into polymer fibers and finally the scaffold itself.

Combined, these two disadvantages lead to the formation of tissue and/or organ engineering scaffolds that do not include any fluorinated compound.

### SUMMARY OF THE INVENTION

It is the object of the present invention to provide a method for the production of labelled scaffolds for tissue and/or organ engineering, which overcomes some of the above mentioned disadvantages.

It is another object of the present invention to provide a method for the production of a labelled scaffold for tissue and/or organ engineering, which leads to a better solubility of highly fluorinated labelling compounds in the polymer mixtures used for the scaffold preparation.

Another object of the present invention is to provide a method for the production of labeled scaffolds, which prevents the evaporation of fluorinated compounds during processing of the mixtures comprising the base material for the scaffold and the fluorinated compound into the scaffold, respectively the fibers forming the later scaffold.

These objects are achieved by the method as set forth in the independent claims. The dependent claims indicate preferred embodiments. In this context it is noteworthy to mention that all ranges given in the following are to be understood as that they include the values defining these ranges.

### BRIEF DESCRIPTION OF THE DRAWINGS

These and other aspects of the invention will be apparent from and elucidated with reference to the embodiments described hereinafter.

Additional details, features, characteristics and advantages of the object of the invention are disclosed in the subclaims, the figures and the following description of the respective figure and examples, which, in an exemplary fashion, show preferred embodiments according to the invention. It is to be understood that the examples are by no means meant as to limit the scope of the invention.

FIG. 1 shows, in an exemplary fashion, a reactive fluorinated surfactant according to the invention

FIG. 2 shows, in an exemplary fashion how the polymerizable end groups can be used to polymerize the reactive fluorinated surfactant of FIG. 1 into a highly fluorinated side chain polymer 20.

FIG. 3 shows, in an exemplary fashion, the reactive fluorinated surfactant 30 of FIG. 1, a second fluorinated compound 31, which is not amphiphilic and a scaffold material 32.

FIG. 4 shows an example of a reactive fluorinated surfactant with amphiphilic character:
2(N-ethylperfluorooctanesulfoamido)ethylacrylate.

FIG. 5 schematically shows how the polymerizable ethylacrylate groups can be used to polymerize the 2(N-ethylperfluorooctanesulfoamido)ethylacrylate

### DETAILED DESCRIPTION OF EMBODIMENTS

According to the invention, a method for the production of a labelled scaffold for tissue and/or organ engineering is provided, comprising at least the following steps:
i.) providing at least one base material for scaffolds;
ii.) providing at least one reactive fluorinated surfactant as imaging label;
iii.) forming a homogeneous mixture of the base material provided in step i) and the reactive fluorinated surfactant provided in step ii.); and
iv.) processing of the mixture formed in step iii.) to form the labeled scaffold.

Basic methods for the production of base materials for scaffolds and the processing of scaffold material are well known in the art, some of them being described herein below.

The said method leads to a better solubility of highly fluorinated labelling compounds in the mixture used for the scaffold processing and therefore prevents phase separation and inhomogeneity of the mixture.

In a preferred embodiment of the present invention, the base material for scaffolds in step i) is a biodegradeable material. Such biodegradable material can be selected from the group consisting of collagen I; collagen II; collagen III; collagen IV; collagen V; collagen VI; collagen VII; collagen VIII; collagen IX; collagen X; elastin; poly(lactide-co-glycolides) (PLGA); polycarpolactone (PCL); polylactic acid (PLA); polyglycolic acid (PGA); tissue culture plastic (TCP); polypropylene fumarate (PPF); poly(ethylene glycol) terephthalate (PEGT); poly(butylene terephthalate); Peptide Hydrogels; polysaccharidic materials, particularly chitosan or glycosaminoglycans (GAGs); hyaluronic acid; particularly in combination with cross linking agents; or any mixtures, copolymers or modifications thereof.

The person skilled in the art has a good understanding of the respective processing, i.e. the polymerization reactions, and the respective monomers used. The term “reactive fluorinated surfactant” (RFS), as used herein, refers to a compound of a multi-component system, in which the components (i.e., at least a polymerizable endgroup, e.g. a monomer; the fluorinated labeling unit and a polar headgroup) may be attached to one another. In a preferred embodiment, the reactive fluorinated surfactant has the general chemical structure of:

A-B—C,

wherein “A” is a highly fluorinated chain, preferable a perfluoro chain; “B” is a polar headgroup; and “C” is a polymerizable endgroup.

The highly fluorinated chain “A” can comprise any fluorine (F) that is suitable as imaging label for use in medical imaging means, like CT, MRI, PET, scintigraphy and/or Ultrasound imaging and the like. Especially the inclusion of perfluoro compounds in tissue and/or organ engineering scaffolds, where the perfluoro atoms serve as contrast agents in Fluor 19 magnetic resonance imaging (Fluor 19-MRI) are preferred. Although fluorine (F) has multiple isotopes, only 19F (Fluor 19) is considered to be stable monoisotopic element.

The inclusion of these labeling compounds in turn enable the in vivo monitoring of the degradation of implanted tissue engineering scaffolds in relation to the growth and remodeling of the newly formed tissue. The inertness and low toxicity of the highly fluorinated compounds forms a clear advantage compared to the lanthanide complexes as applied in prior art.

The polar headgroup “B” can be any chemical compound possessing together with the highly fluorinated chain “A” the required amphiphilic properties of the reactive fluorinated surfactant.

“Amphiphilic” is a term describing a chemical compound possessing both hydrophilic and hydrophobic properties. As a result of having both hydrophobic and hydrophilic structural regions, the reactive fluorinated surfactant may dissolve in water and to some extent in non-polar organic solvents and therefore enables the formation of homogenous mixture of the polymer mixture used for the preparation of the scaffold.

In a preferred embodiment the highly fluorinated chain “A” possesses the hydrophobic properties, whereas the polar headgroup “B” provides the hydrophilic group, like anionic charged groups (e.g. carboxylates: RCO2−; sulfates: RSO4−; sulfonates: RSO3−; phosphates: the charged functionality in phospholipids) or cationic charged groups (e.g. amines: RNH3+), wherein the hydrophobic part of the molecule is represented by an R; or polar, uncharged groups (e.g. alcohols with large R groups, such as diacyl glycerol (DAG), and oligoethyleneglycols with long alkyl chains).

The polymerizable endgroup “C” can be any monomer or polymer that is capable to polymerize the reactive fluorinated surfactant during and/or after the processing of the homogenous mixture comprising the scaffold base material and the reactive fluorinated surfactant

In another embodiment of the present invention the 3 parts “A”, “B” and “C” of the reactive fluorinated surfactant are prior to their attachment to each other selected from a not limiting group of single substances, whereas “A” is a perfluoroalkyl; “B” is selected from the group consisting of carboxylates, sulfates, sulfonates and phosphates; and “C” is selected from the group consisting of acrylates, methacrylates, acrylamides, epoxides and oxetanes.

Synthesis of the reactive fluorinated surfactant in whole comprising the parts “A”, “B” and “C” or attachment of the single parts “A”, “B” and “C” to each other is common knowledge to any person skilled in the art and herewith incorporated by reference.

In a preferred embodiment the reactive fluorinate surfactants is a
2(N-alkyl-perfluoro-alkansulfoamido)alkyl(meth)acrylate, preferably a
2(N-ethylperfluororoctanesulfoamido)methylacrylate, most preferably a
2(N-ethylperfluororoctanesulfoamido)ethylacrylate.

In yet another embodiment the polymerizable endgroup “C” of the reactive fluorinated surfactant polymerize during and/or after the processing of the mixture in step iv) into a highly fluorinated sidechain polymer.

It is notable that the reactive fluorinated surfactant can be incorporated within and/or on the scaffold. In particular, the reactive fluorinated surfactant can be localized both on the outer surface of the polymer material/polymer fibers forming the scaffold and/or between the polymer material/fibers forming the scaffold. Due to the amphiphilic character of the reactive fluorinated surfactant, it will preferentially be located at the polymer/air interface and/or it will be present in the polymer fibers as micellular structures.

According to the polymerization of the highly fluorinated surfactant into highly fluorinated sidechain polymers evaporation of the imaging enhancement agent is prohibited and the mobility of the fluorinated compounds in the polymer matrix of the scaffold is strongly reduced.

For the purpose of performing the polymerization of the polymerizable endgroups “C” it is advantages to co-dissolve an appropriate photoinitiator. The photoinitiator can be selected from the group consisting of Alpha, alpha-dimethoxy-alpha-phenylacetophenone; 1-Hydroxy-cyclohexyl-phenyl-ketone; 2-Hydroxy-1-[4-(2-hydroxyethoxy)phenyl]-2-methyl-1-propanone; 2-Methyl-1-[4-(methylthio)phenyl-2-(4-morpholinyl)-1-propanone; Diphenyl (2,4,6-trimethylbenzoyl)-phosphine oxide and 2-Hydroxy-2-methyl-1-phenyl-1-propanone.

In a further embodiment the homogenous mixture of base material and highly fluorinated surfactant further comprises another highly fluorinated compound without amphiphilic character. This further compound can be perfluorooctylbromide (PFOB).

In yet another preferred embodiment of the present invention the processing of the mixture in step iv) to form the scaffold is based on a method selected from the group consisting of

a) electrospinning,

b) rapid prototyping,

c) knitting, and/or

d) phase separation.

Electrospinning is a method for generating ultrathin fibers from materials such as polymers, composites, and others. Nano fibers of both solid and hollow interiors (“nanotubes”) can be formed as well. The thin fibers are produced by uniaxial stretching of a viscoelastic jet derived from a polymer solution or melt by applying high voltages. The fibers are deposited on a flat surface and lead to a complex meshwork which afterwards can be shaped. The electrospinning process for making scaffolds is known in the art as described by Van Lieshout et al. (2006).

The term “Rapid prototyping” as used herein, refers to methods for the construction of physical objects using solid freeform fabrication, i.e., without a mould. Rapid prototyping takes virtual designs from computer aided design (CAD) or animation modeling software, transforms them into thin, virtual, horizontal cross-sections and then creates each cross-section in physical space, one after the next until the model is finished. Table 7 gives a non-limiting overview of some rapid prototyping methods which can be used in the context of the present invention.

TABLE 1

| Prototyping Technologies | Base Materials (examples) |
|---|---|
| Selective laser sintering (SLS) | thermoplastics, metals powders |
| Fused Deposition Modeling (FDM) | thermoplastics, eutectic metals. |
| Stereolithography (SLA), or three | photopolymerizable polymers |
| dimensional lithography | |
| Electron Beam Melting (EBM) | titanium alloys |
| 3D Printing (3DP) | various |
| Solid ground curing (SGC) | photopolymerizable polymers |

Knitting is a method well known in the art. In scaffold production, it allows the production of an open structure that is mechanically reliable. An advantage of knitting is the complex geometries that can be produced, e.g., branched prostheses used for aortic arch replacements among the materials that have been used for knitting are Dacron, but also carbon fibers as well as polymers like polycaprolactone. The knitting process for making scaffolds is as well described by Van Lieshout et al. (2006).

Phase separation comprises different approaches, them being immersion precipitation, solid-liquid phase separation, liquid-liquid phase separation, polymerization-induced phase separation and particularly thermally induced phase separation (“TIPS”). The latter is a method that requires the use of a solvent with a low melting point that is easy to sublime. This can for example be done with dioxane as a solvent for polylactic acid. Phase separation is then induced by addition of a small quantity of water, which leads to the formation of a polymer-rich phase and a polymer-poor phase. The mixture is then cooled below the solvent melting point, and vacuum-dried, to sublime the solvent in order to obtain a porous scaffold.

Other methods for the production of scaffolds which fall under the scope of the present invention comprise at least one selected from the group consisting of
gas foaming with injected gas, CaCO₃ or NH₄HCO₃
sintering of microspheres
Super critical fluid technology
Particulate leaching
Emulsification
Freeze drying
Solvent casting
Extrusion
Production of Nonwovens
Weaving
Fibre bonding
Membrane lamination
Hydrocarbon templating
Solid Freeform Fabrication techniques
Mould casting, and/or
Microrobotics/micro machining

In another preferred embodiment of the present invention, a homogeneous mixture for processing a labelled scaffold for tissue and/or organ engineering comprising at least one base material for scaffolds and at least one reactive fluorinated surfactant is provided. The homogenous mixture comprises ≧0.01 to ≦10% w/v, preferably ≧0.05 to ≦5% w/v, more preferably ≧0.1 to ≦2% w/v, most preferably ≧0.2 to ≦1.0% w/v of the reactive fluorinated surfactant.

It is notable that the mixture of the scaffold base material and the reactive fluorinated surfactant can be either a suspension or a solution.

The solvent for the homogeneous mixture of the base material for the scaffolds and the reactive fluorinated surfactant is an organic solvent. This solvent can be selected from the group consisting of dichloromethane, trichloromethane, toluene, xylene(s) and tetrahydrofuran.

In a preferred embodiment the homogeneous mixture further comprises a highly fluorinated compound and/or a photoinitiator.

Other preferred embodiments of the invention are targeted to a scaffold for tissue and/or organ engineering that comprises at least one reactive fluorinated surfactant. The scaffold comprises ≧0.001 to ≦10% w/w, preferably ≧0.01 to ≦5% w/w, more preferably ≧0.1 to ≦2% w/w, most preferably ≧0.2 to ≦1% w/w of the fluorine.

As used herein, % w/v refers to percent weight in volume and expresses the number of g of a constituent in 100 ml of solution.

As used herein, % w/w refers to percent weight in weight, and expresses the number of g of a constituent in 100 g of solution or mixture.

Other preferred embodiments of the invention are targeted to a scaffold useful for the manufacture of a tissue and/or organ, characterized in that said scaffold comprises at least one reactive fluorinated surfactant.

Said scaffold is, in a preferred embodiment, obtainable by a method according the invention.

Said scaffold is, in another preferred embodiment being used for the production of at least one tissue and/or organ selected from the group consisting of

a) artificial heart valves,

b) vascular grafts,

c) skin,

d) nervous tissue,

e) organs,

f) bladder,

g) blood vessels,

h) cartilage tissue, and/or

i) bone tissue.

Furthermore, the use of such scaffold for the manufacture of a tissue and/or organ is provided, as well as a tissue and/or organ comprising such scaffold. Said tissue and/or organ is preferably selected from at least one of the group consisting of

a) artificial heart valves,

b) vascular grafts,

c) skin,

d) nervous tissue,

e) organs,

f) bladder,

g) blood vessels,

h) cartilage tissue, and/or

i) bone tissue.

The invention is broadly applicable in the field of tissue and/or organ engineering to follow post-implantation remodelling of tissue engineering constructs and in-vivo degradation of scaffolds and/or scaffold materials in relation to the growth and remodelling of newly formed tissue. The monitoring can be performed with medical imaging means, like CT, MRI, PET, scintigraphy and/or Ultrasound imaging. Clinical MRI scanners, equipped with the necessary coils and software for 19 Fluor MRI, are preferred.

### DEFINITIONS

The term “reactive fluorinated surfactant” (RFS), as used herein, refers to a compound of a multi-component system, in which the components (i.e., at least a polymerizable endgroup, e.g. a monomer; the fluorinated labeling unit and a polar headgroup) may be attached to one another.

The term “tissue and/or organ engineering”, as used herein, refers to an interdisciplinary field that applies the principles of engineering and life sciences toward the development of biological substitutes that restore, maintain, or improve tissue function or a whole organ. It comprises the use of a combination of cells, engineering and materials methods, and suitable biochemical and physio-chemical factors, to improve or replace biological functions, particularly tissues and/or organs.

This includes the repair or replacement of portions of, or whole, tissues and/or organs (i.e., bone, cartilage, blood vessels, bladder, etc.). sometimes resulting in artificial organs and/or tissues, like an artificial pancreas, or a bioartificial liver. Tissue engineering requires, in most cases, a scaffold and living cells to colonize the former.

The term “scaffold”, as used herein, relates to a three dimensional matrix on which cells are grown. These matrices are often critical, both ex vivo as well as in vivo, to recapitulating the in vivo milieu and allowing cells to influence their own microenvironments. The term also refers to both: the complete scaffold or scaffold materials that will be further processed into scaffolds.

Scaffolds usually serve at least one of the following purposes:
Allow cell attachment and migration
Deliver and retain cells and biochemical factors
Enable diffusion of vital cell nutrients and expressed products
Exert certain mechanical and biological influences to modify the behaviour of the cell phase
Provide mechanical support for the newly developing tissue

To achieve the goal of tissue reconstruction, scaffolds must meet some specific requirements. A high porosity and an adequate pore size are necessary to facilitate cell seeding and diffusion throughout the whole structure of both cells and nutrients. Biodegradability is often an essential factor in case the scaffolds are supposed to be absorbed by the surrounding tissues over time without the necessity of a surgical removal. The rate at which degradation occurs has to coincide as much as possible with the rate of tissue formation. This means that while cells are fabricating their own natural matrix structure around themselves, the scaffold provides structural integrity within the body, and eventually it will break down leaving the so called neotissue, i.e., newly formed tissue which will take over the mechanical load.

Cells as used for tissue engineering comprise, among others, fibroblasts and/or keratocytes (for skin replacement or repair), chondrocytes (for cartilage replacement or repair), stem cells (large variety of potential tissues to be replaced, or repaired), pluripotent cells (large variety of potential tissues to be replaced, or repaired), cardiac stem cells (for the repair or replacement of cardiac tissue), endothelial stem cells (for the repair or replacement of vascular tissue), valve stem cells (for the repair or replacement of heart valves), and so forth.

The cells used comprise, in a preferred embodiment, extended telomeres, in order to increase their dividing potential and/or lifetime, which is restricted, in non-modified cells, by the so-called Hayflick limit.

Particularly preferred, the cells used are autologous cells, i.e., cells which are genetically compatible with the recipient of the tissue or organ produced therewith. This is basically the case if the cells are derived from the same subject to which the cells are applied (i.e., donor and recipient are the same person), or in case donor and recipient are close relatives.

The term “base material for scaffolds”, as used herein, refers to any substance which is useful for the construction of scaffolds, prior to their processing into a scaffold, e.g. by electrospinning, or into scaffold materials like polymer fibers that will be further processed.

The term “labelled scaffold”, as used herein, refers to a scaffold with a marker substance, e.g. an imaging agent.

The term “imaging agent”, as used herein, refers to an agent, i.e., a molecule, which can be made visible by means of an imaging apparatus, like an X-ray, a computer tomograph (CT, particularly spectral CT, a Magnetic Resonance Imager (MRI), a sonograph, a positron emission tomograph (PET) and/or a scintigraph (see table 1). The visualization is particularly useful when an embodiment labelled with said labelling agent is implanted into the human body. In this case, on would speak of in situ visualization, or in vivo visualization. Frequently, the said imaging agents are also termed “contrasting agents”.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### DISCUSSION OF THE FIGURES

The following figures illustrate schematically the essential aspects of the invention.

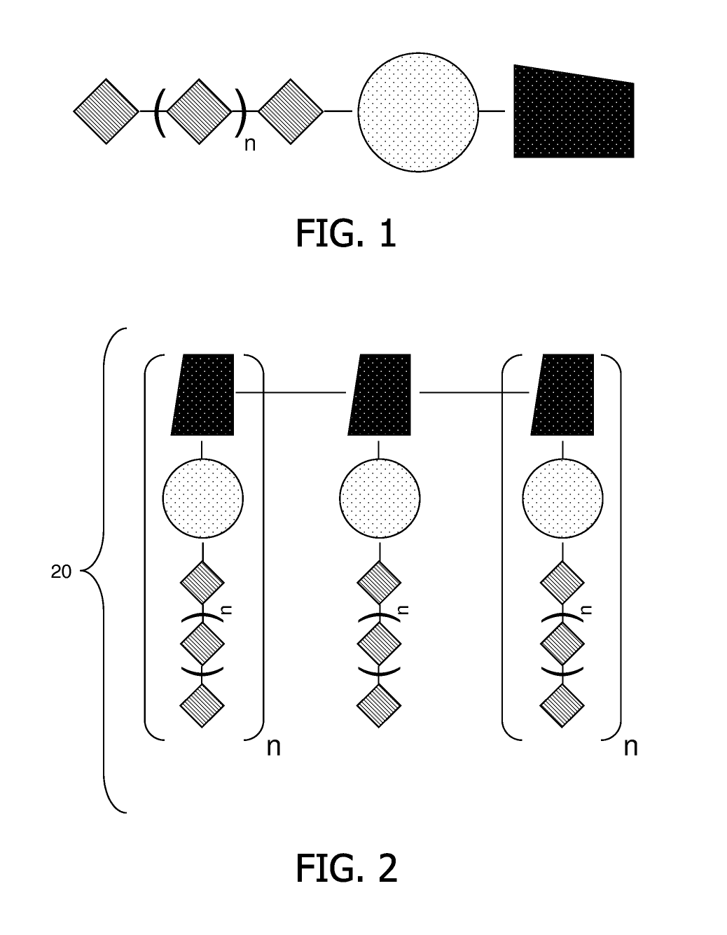

FIG. 1 shows, in an exemplary fashion, a reactive fluorinated surfactant according to the invention comprising a highly fluorinated (perfluoro) chain of variable length 10, linked to a polar head group 20 which in turn is linked to a polymerizable end group 30. Due to this arrangement the compound has an amphiphilic character. This characteristic greatly facilitates the formation of a homogenous mixture, when the reactive fluorinated surfactant 30 is added to the mixture, which forms the later scaffold.

FIG. 2 shows, in an exemplary fashion how the polymerizable end groups can be used to polymerize the reactive fluorinated surfactant 30 of FIG. 1 into a highly fluorinated side chain polymer 20.

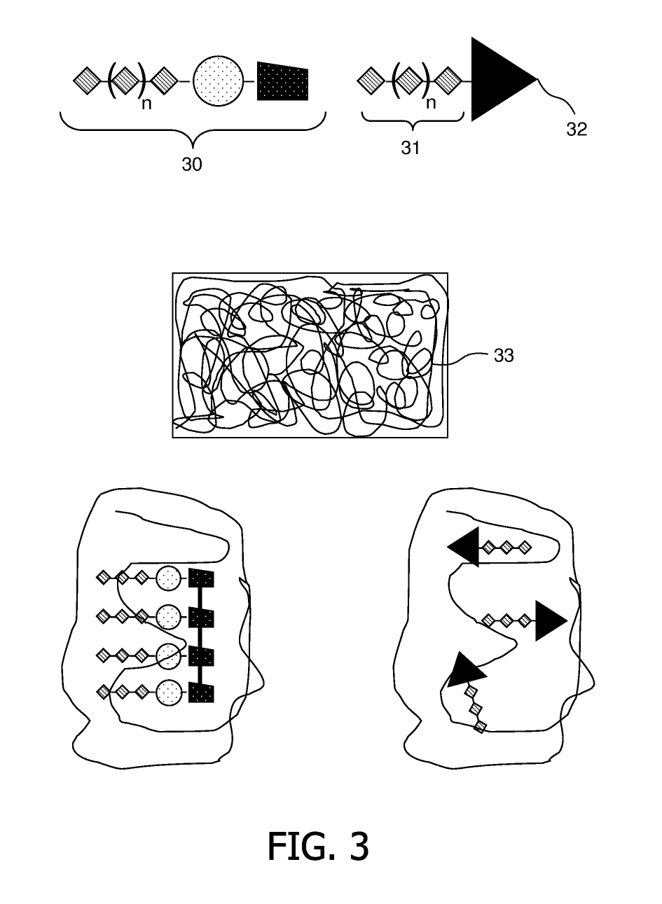

FIG. 3 shows, in an exemplary fashion, the reactive fluorinated surfactant 30 of FIG. 1, a second fluorinated compound 31, which is not amphiphilic and a scaffold material 32. When the commonly applied non-reactive fluorinated compound is added to the scaffold the individual molecules remain separate. This leads to a high mobility of the compound in the scaffold material 32 and may cause fast evaporation during scaffold processing.

In case of the reactive fluorinated surfactant 30 however, the polymerizable end groups can be used to polymerize the reactive fluorinated surfactant 30 of into a highly fluorinated side chain polymer, thus reducing mobility in the scaffold material 32 and avoiding fast evaporation of the fluorinated surfactant 30 during scaffold processing.

FIG. 4 shows an example of a reactive fluorinated surfactant 30 with amphiphilic character 2(N-ethylperfluorooctanesulfoamido)ethylacrylate. The amphiphilic character of the compound enables the formation of a homogenous suspension when 2(N-ethylperfluorooctanesulfoamido)ethylacrylate is added to the mixture used for the preparation of the scaffold.

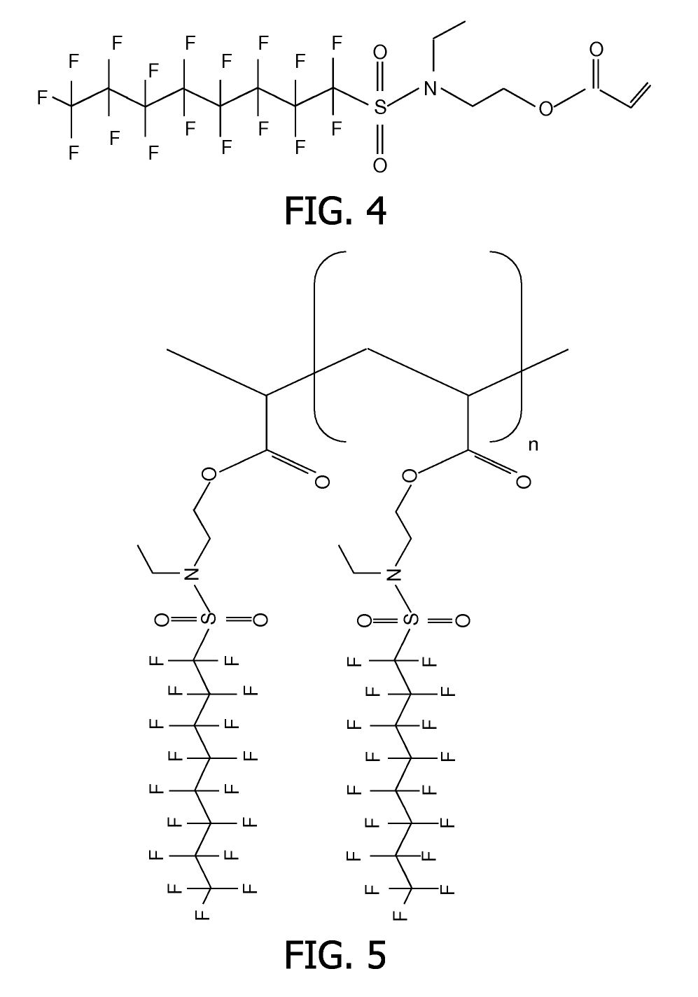

FIG. 5 schematically shows that 2(N-ethylperfluorooctanesulfo amido)ethylacrylate how the polymerizable ethylacrylate groups can be used to polymerize 2(N-ethylperfluorooctanesulfoamido)ethylacrylate into a highly fluorinated side chain polymer.

### EXAMPLES

In order to demonstrate the essential features of the invention two polymer solutions have been prepared for scaffold construction by means of electrospinning. Electrospinning is a simple method for generating ultrathin fibers from materials such as polymers, composites, and others. Nanofibers of both solid and hollow interiors can be formed. The thin fibers are produced by uniaxial stretching of a viscoelastic jet derived from a polymer solution or melt by applying high voltages. The fibers are deposited on a flat surface and lead to a complex meshwork which afterwards can be shaped.

The first electrospin solution contains perfluorooctylbromide (PFOB) as commonly used in prior art. The second solution contains, next to the PFOB a small amount of the reactive fluorinated surfactant 2(N-ethylperfluorooctanesulfo amido)ethylacrylate.

Electrospin Solution 1:

3.4 grams of polycaprolactone was dissolved in 16.6 grams chloroform by stirring for 24 hours at room temperature. Directly before electrospinning, 170 mg of PFOB was added and the mixture was stirred for 5 minutes before the mixture was transferred to the electrospin equipment.

Electrospin Solution 2:

3.4 grams of polycaprolactone was dissolved in 16.6 grams dichloromethane by stirring for 24 hours at room temperature. Directly before electrospinning, 170 mg of PFOB and 17 mg of 2(N-ethylperfluorooctanesulfoamido)ethylacrylate were added and the mixture was stirred for 5 minutes before the mixture was transferred to the electrospin equipment.

After processing, the scaffolds obtained from the two electrospin solutions were analysed for fluor content after destruction of the sample by Schöniger combustion. An amount of sample (approximately 5 mg, in duplicate) was brought onto a clean filter, which was totally combusted in a closed flask in the presence of oxygen. The reaction products were taken up in a mixture of NaOH and water. After destruction the solution was diluted and the amount of Fluoride was determined using Ion Chromatography (Dionex ICS3000) with conductivity detection.

Using this technique, the sample was introduced into a mobile phase via an injection loop. Subsequently the sample was pumped with the eluent through an analytical ion-exchange column. Due to differences in affinity of the sample ions towards the mobile phase and ion-exchange material the ions traveled with different velocities through the analytical column. As a result the various ions were separated in time and detected one by one with a conductivity detector. Quantification was performed by comparison of the measured peak heights with those produced by standard solutions. Generally, the inaccuracy of certain analyses was estimated to be 5-10% relative.

The results showed that the scaffolds obtained from electrospin solution 1 do not contain a detectable amount of fluoride while the scaffolds obtained from solution 2 contain 0.4% weight fluoride/weight scaffold. The measured fluoride in the s content in scaffolds obtained from electrospin solution 2 matches the original amount of 2(N-ethylperfluorooctanesulfoamido)ethylacrylate the spin solution 2.

### REFERENCES

van Lieshout, M. I., C. M. Vaz, M. C. Rutten, G. W. Peters, and F. P. Baaijens, Electrospinning versus knitting: two scaffolds for tissue engineering of the aortic valve. J Biomater Sci Polym Ed. 17:77-89, 2006

1. A method for the production of an labelled scaffold for tissue and/or organ engineering is provided, comprising at least the following steps:
i) providing at least one base material for scaffolds;
ii) providing at least one reactive fluorinated surfactant as imaging label;
iii) forming a homogeneous mixture of the base material provided in step a) and the reactive fluorinated surfactant provided in step ii);
iv) processing of the mixture formed in step iii) to form the labelled scaffold.

2. The method according to claim 1, wherein the reactive fluorinated surfactant provided in step ii) has the general chemical structure of:
A-B—C,
wherein “A” is a highly fluorinated chain, preferable a perfluoro chain;
“B” is a polar headgroup; and “C” is a polymerizable endgroup.

3. The method according to claim 2, wherein “A” is a perfluoroalkyl; “B” is selected from the group consisting of carboxylates, sulfates, sulfonates and phosphates; and “C” is selected from the group consisting of acrylates, methacrylates, acrylamides, epoxides and oxetanes.

4. The method according to claim 1, wherein the reactive fluorinated surfactant is a
2(N-alkyl-perfluoro-alkansulfoamido)alkyl(meth)acrylate.

5. The method according to claim 2, wherein the polymerizable endgroup “C” of the reactive fluorinated surfactant polymerize during and/or after the processing of the labelled scaffold material and/or labelled scaffold in step iv) into a highly fluorinated sidechain polymer.

6. The method according to claim 1, wherein the mixture further comprises a photoinitiator.

7. The method according to claim 1, wherein the mixture further comprises a highly fluorinated compound without amphiphilic character.

8. A homogeneous mixture for processing a labelled scaffold for tissue and/or organ engineering comprising at least one base material for scaffolds and at least one reactive fluorinated surfactant.

9. A homogenous mixture according to claim 8 comprising ≦0.01 to ≦10% w/v of the reactive fluorinated surfactant.

10. A scaffold for tissue and/or organ engineering, characterized in that it comprises at least one reactive fluorinated surfactant.

11. The scaffold according to claim 10, wherein the scaffold comprises ≧0.001 to ≦10% w/w of the fluorine.

12. The scaffold according to claim 10, which is being used for the manufacture of a tissue and/or organ.

13. The scaffold according to claim 10, obtainable by a method according to claim 1.

14. A tissue and/or organ comprising a scaffold according to claim 10.
